(19) United States
(12) Patent Application Publication
(10) Pub. No.: US 2016/0271004 A1
(43) Pub. Date: Sep. 22, 2016

(54) FEEDBACK ELECTRO HEMOSTATIC COMPRESSION PAD

(71) Applicant: SANKO ÖZEL EGITIM HIZMETLERI A.S., Gaziantep (TR)

(72) Inventors: Burak ERDINC, Gaziantep (RS); Orhan CEVIK, Gaziantep (TR); Burak ERKILIC, Gaziantep (TR)

(73) Assignee: SANKO ÖZEL EGITIM HIZMETLERI A.S., Gaziantep (TR)

(21) Appl. No.: 14/777,628

(22) PCT Filed: Mar. 20, 2014
(86) PCT No.: PCT/TR2014/000086

§ 371 Date: Sep. 16, 2015

(30) Foreign Application Priority Data
Mar. 20, 2013 (TR) 2013/03357

(51) Int. Cl. A61H 9/00; A61B 5/00; A61B 5/021

(52) U.S. Cl. CPC A61H 9/0078; A61B 5/021; A61B 5/4836; A61B 5/486; A61H 2201/5071

(57) ABSTRACT

A feedback electro hemostatic compression pad developed to decrease hospitalization duration after angiography and to provide a more comfortable angiography, and which comprises a foldable (2) paddle (1) when necessary and a compressing conservation balloon (4), a micro air pump (5) which provides inflating of the conservation balloon (4) after the instruction sent according to the blood pressure values sensed with sensors and stabilizing thereof on the determined blood pressure, a control panel (6) controlling said micro air pump (5) and an air vent (3) which decreases the air in conservation balloon (4) according to the changing blood pressure values and vents the air at the end of the operation.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT Not applicable.

### REFERENCE TO AN APPENDIX SUBMITTED ON COMPACT DISC Not applicable.

### BACKGROUND OF THE INVENTION

1. Field of the Invention

The invention, in the medical field, is related to a hemostatic assembly to staunch the leaking blood with the required compression to angiography area of the body after angiography.

The invention is especially related to feedback electro hemostatic angiography compression pad which is provides the required compression electronically, and prevents hematoma, and which is developed to reduce duration of hospitalization after angiography and provides a more comfortable angiography.

2. Description of Related Art Including Information Disclosed Under 37 CFR 1.97 and 37 CFR 1.98

Cardiac catheterization and angiography are diagnosis methods, not treatment. Those are based on scanning the cardiac cavity and coronary arteries during dyeing with a contrast agent, i.e. kind of medical pigments, and taking motion images using X-rays. Parameters obtained are very valuable for the treatment process and in many patients it is the key determinant for the treatment strategy. Ratio of success of the mentioned operations is above 99% by means of recent technological conditions and knowledge.

The patient should be starved before cardiac catheterization and angiography between 4-12 hours. The medicines can be taken with very few water. The angiography area of the body should be shaved for sterilization before taking the patient to the catheterization laboratory. Tranquillizer can be medicated when necessary. Part of the groin or the arm that the medical intervention will be made, is anesthetized and the puncture hole is opened with cannulating the artery in this area. Pressure is recorded after reaching the cardiac cavity with a thin tube (catheter) made of plastic like substance; coronary artery is monitored injecting the contrast agent and the dynamic shots are recorded. This medical process takes about 20-30 minutes with the recent technology.

The catheter entering the artery cannot be withdrawn immediately and should be waited for 4-6 hours. During this period the patient should lie still without moving his leg or the angiography area. After 4-6 hours, the catheter will be withdrawn and bleeding will be staunched with 15-20 minutes of hand compression and placing sandbag for 4-6 hours. Accordingly, the patient on whom diagnostic coronary angiography is performed with vascular access should lie still for 6 hours and the patient on whom therapeutic interventions such as placing a balloon or stent are performed should lie on his/her bed for approximately 12 hours without moving the angiography area.

In the literature, there are some previous applications in this field. One of them is the application No. 2011/08853 and titled “Hemostatic Allergic Balloon Assembly for Radial Region”. According to the abstract of the application, it is a hemostatic balloon assembly for radial region which comprises a cavity, air inlet connector placed on one side of the cavity on the wristband and a balloon providing compression to the bleeding area by means of inflating on the other side; air inlet balloon holder connected to the balloon; valve connected to said air inlet connector to inflate and vent the balloon; wherein it further comprises wristband surrounding the wrist of the patient and coating which is made of wood pulp covering the balloon, accelerates the healing of the postoperative scars, provides sterilization, prevents possible skin irritations such as allergy, rush etc.

In the abstract of another application in the literature, WO 2008/033401 a pad of elastomeric material which is developed to prevent bleeding and leaking and staunches thereof on the affected surface area with low pressure application of the pad to the skin, used especially in the inguinal angiography is disclosed.

Consequently, hemostatic compression systems after angiography are improving and new embodiments are required to bring new solutions to the present systems and eliminate the disadvantages mentioned above.

### BRIEF SUMMARY OF THE INVENTION

The present invention is especially related to feedback electro hemostatic angiography compression pad which meets the requirements above, eliminates all the disadvantages and brings new advantages, and which provides the required compression electronically and prevents hematoma, and is developed to reduce duration of hospitalization after angiography and provides a more comfortable angiography.

One objective of the invention is to reduce the time of lying still on the bed after the angiography, before withdrawing the properly placed catheter.

Another objective of the invention is to inflate the balloon to the determined pressure after sensing the blood pressure with the sensors provided in the device, instructing the micro air pump and to stabilize thereof after reaching the required pressure.

Another objective is to prevent possible leaks from the incision area by increasing or decreasing the air in the balloon according to blood pressure change when necessary. With this method post angiography period becomes more secure and leak is prevented as well as hematoma.

In order to achieve all the objects mentioned above and to be understood from the detailed description below, the present invention which is developed to reduce hospitalization duration after angiography and provide more comfortable angiography; is related to feedback electro hemostatic compression pad comprising foldable paddle and compressing conservation balloon, wherein it further comprises micro air pump which provides inflating of the conservation balloon at a determined pressure and stabilizing thereof at the determined pressure after the instruction sent according to the blood pressure values sensed with sensors, control panel which controls the micro air pump, and air vent which decreases the air in the conservation balloon according to the changing blood pressure values and vents the air at the end of the operation.

In the preferred embodiments of the invention, pad dashboard enabling observation of the angiography area is included.

Structural features, characteristics and all advantages of the invention will be understood more clearly with the below figures and detailed description written with reference to these figures. The examination should be made by taking these figures and detailed description into account.

### BRIEF DESCRIPTION OF THE DRAWINGS

In order to understand the embodiments and advantages o the invention with its additional elements, it should be examined with the figures described below.

FIG. 1: Perspective of feedback electro hemostatic compression pad.

FIG. 2: Side view of feedback electro hemostatic compression pad.

FIG. 3: Back view of feedback electro hemostatic compression pad.

The drawings need not to be scaled and unnecessary details to understand the invention may have been ignored. Further, the elements which are at least considerably identical or at least considerably identical in function are mentioned with the same number.

### REFERENCE NUMBERS

1. Pad Paddle

2. Folded Form of the Padded Paddle

3. Pad Air Vent

4. Pad Air Conservation Balloon

5. Pad Micro Air Pump

6. Pad Micro Air Pump Control Keys

7. Pad Dashboard

### DETAILED DESCRIPTION OF THE INVENTION

In this detailed description, the preferred embodiments of the feedback electro hemostatic compression pad according to the present invention are described only for the subject to be understood better without any limiting effect.

With the feedback electro hemostatic compression pad according to the invention, time of waiting for long on the bed after the angiography has been reduced. With the feedback electro hemostatic compression pad, before the catheter is withdrawn, the pad is inflated according to the blood pressure sensed by the sensors, catheter is withdrawn while the required compression is supplied and the patient experiences comfortable angiography by observing hemostasis.

The patient who had coronary angiography can be discharged within 4 hours and the heparinized (with blood coagulants) patients who had treatments like placing a stent or balloon can be discharged within a total of 6 hours, by using feedback electro hemostatic compression pad according to the invention upon waiting for 2 hours with catheter. Hospitalization duration of the patient who had coronary angiography by using feedback electro hemostatic compression pad is 90 minutes after the bleeding is stopped and in the next 2 hours after venting of the air, the patient can move and relieve his/herself. It is a very safe and effective method. It prevents occurring of hematoma.

Operating logic of the feedback electro hemostatic compression pad is in the form of inflating the pad with the help of the sensors according to the blood pressure before the catheter is withdrawn and venting the air immediately with only one button when desired. In the balloon occlusion method to be applied to the incision area, the blood pressure is sensed by sensor placed between the balloon and the incision area. Said sensor is integrated to the system. Information from the sensor is processed by microcontrollers and the decision is made. The decision information is sent from the microcontroller to the air pump (5) and the compression is increased or decreased accordingly. The dimensions and weight of the developed product are determined considering the criteria such as ease of use.

It consists of a balloon assembly providing the required compression electronically, and preventing hematoma in order to staunch the bleeding after the angiography. It is preferably in different dimensions of 12-27-37-52-66 cm and can be used easily even for overweight patients.

This invention mainly aims to occlude the puncture hole after stent operations performed with vascular access.

In state of art, high dose of anticoagulants are used during the medical intervention. After the intervention the catheter cannot be withdrawn immediately and four or six hours should be elapsed. Meanwhile, the patient should lie still without moving his/her leg. After 4-6 hours, the catheter will be withdrawn and bleeding will be staunched with 15-20 minutes of hand compression arid placing sandbag for 4-6 hours. Accordingly, the patient on whom coronary angiography is performed should lie still for 6 hours and the patient on whom therapeutic interventions such as placing a balloon or stent are performed should lie on his/her bed for approximately 12 hours without moving the angiography area.

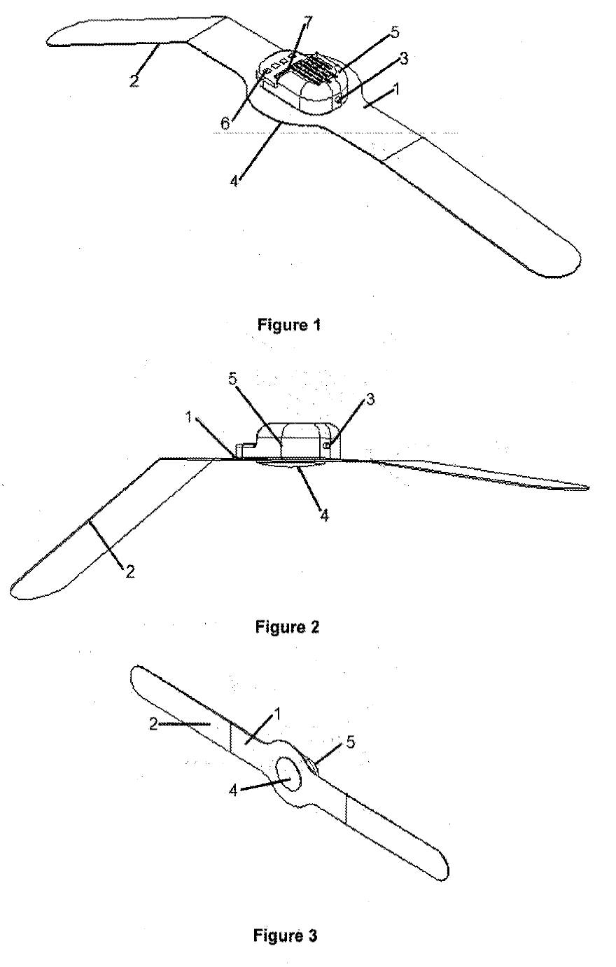

With the developed feedback electro hemostatic compression pad, the time of waiting for long on the bed has been reduced to less time. In FIG. 1 paddle of pad (1) and folded form of that paddle (2) are observed. Said paddle (1) is flexible and can adapt to the applied body part as seen. Thanks to the feedback electro hemostatic compression pad, before the catheter is withdrawn in the angiographies, the air conservation balloon (4) is inflated by means of micro air pump (5) according to the blood pressure sensed by the sensors. The balloon (4) is inflated in accordance with the previously measured reference values. Said micro air pump (5) is controlled by air pump control keys (6). While the required compression is supplied, catheter is withdrawn and hemostasis is observed by means of pad dashboard (7) and the patient has a comfortable angiography. Then the air in conservation balloon (4) is vented by pad air vent (3). According to the change of blood pressure values, excess air in conservation balloon (4) is vented by pad air vent (3). According to the blood pressure, conservation balloon (4) is inflated by micro air pump (5) if necessary.

The most important point after angiography is to compress the puncture hole. The main intervention is to occlude the 3 mm puncture hole opened in order for the catheter to be inserted into the vein. Problems arising from the puncture hole are due to the inability to occlude said hole. The vein used in angiography from groin is a major vein. There are bones there behind and muscle and fat tissues at the back. Especially for overweight women, there is a good compression because of the fat. In angiography from the wrist, as there is bone under the vein, with the help of a special product like wristband, bleeding is staunched and the puncture hole is occluded immediately, the feedback electro hemostatic compression pad strips designed to staunch the bleeding in inguinal angiography and angiography from wrist are very effective, safe and low costing methods.

1. A feedback electro hemostatic compression pad which is developed to decrease hospitalization duration after angiography and to provide a more comfortable angiography, and which comprises a foldable paddle when necessary and a compressing conservation balloon, characterized in comprising:
a micro air pump which provides inflation of the conservation balloon after the instruction sent according to the blood pressure values sensed with sensors and stabilizing thereof on the determined blood pressure;
a control panel controlling said micro air pump; and
an air vent which decreases the air in the conservation balloon according to the changing blood pressure values and vents the air at the end of the operation.

2. Feedback electro hemostatic compression pad according to claim 1, characterized in comprising a pad dashboard ensuring observation of the angiography area.
